(19) United States
(12) Patent Application Publication
(10) Pub. No.: US 2026/0297492 A1
(43) Pub. Date: Oct. 1, 2026

(54) System And Method For Preparation And Sterilization Of Buffer Or Cell Culture Medium

(71) Applicant: Yingqing Huang, Buffalo Grove, IL (US)

(72) Inventor: Yingqing Huang, Buffalo Grove, IL (US)

(21) Appl. No.: 19/699,851

(22) Filed: Jun. 5, 2026

Related U.S. Application Data
(62) Division of application No. 18/490,551, filed on Oct. 19, 2023.

(51) Int. Cl. C12M 1/26; B01D 61/18; B01D 61/22; B01D 69/02; C12M 1/36

(52) U.S. Cl. CPC C12M 33/14; B01D 61/18; B01D 61/22; B01D 69/02; C12M 41/48; B01D 2311/06; B01D 2313/501; B01D 2315/10; B01D 2317/025; B01D 2317/08; B01D 2325/02833; B01D 2325/02834

(57) ABSTRACT

Disclosed herein is a modular system for robust, efficient preparation of cell culture media and buffers, comprising a) a tangential flow filtration (TFF) module for initial mixing, dissolution, and pre-filtration of concentrated stock solutions to remove particulates, bacteria, enabling handling of lower quality raw materials; b) optional intermediate holding tanks for temporary storage, sampling, and quality control; c) a final mixing module diluting concentrated, pre-filtered stock solutions before terminal sterile filtration. The multi-step approach with TFF pre-filtration provides smaller equipment footprint, lower capital costs, and continuous/semi-continuous buffer production capability versus traditional batch preparation. Process automation and optimization of intermediate dilution ratios based on TFF process rate enable seamless operation, feeding of multiple staggered bio-reactors, and significant operating cost savings. The invention provides more robust, flexible, and cost-efficient cell culture media and buffer preparation suitable for large-scale bio-processing.

### FIELD OF THE INVENTION

The present invention generally relates to improved systems and methods for efficient, robust, and cost-effective preparation and sterile filtration of cell culture media and buffers at large scales for bio-pharmaceutical manufacturing and bio-processing applications.

### BACKGROUND OF THE INVENTION

The biopharmaceutical and emerging cell-cultured meat industries are in dire need of scalable and cost-effective solutions for large-scale cell culture media production. Conventional methodologies rely on the utilization of stirring tanks that extend to the total final volume for the purposes of initial mixing and dissolution before sterile filtration. This necessitates a substantial upfront investment in expansive equipment and facilities, often equivalent in size to the bioreactor volumes themselves.

For the production of high-value biologics, the costs associated with these methods are manageable, especially when operating at relatively smaller scales, typically up to 20,000 liters (L). However, in the context of large-scale bio-manufacturing applications, such as the production of cell-cultured meats, where the requisite volumes exceed 200,000 L, the traditional media preparation processes become prohibitively expensive. This widening cost disparity underscores the immediate demand for innovative processes aimed at mitigating both capital and operational expenses.

Furthermore, recent advancements in process intensification in bioproduction have significantly heightened the requirements for robust, high-yield downstream processes. Clarification, in particular, poses a formidable challenge, as the elevated cell densities lead to the accumulation of cell debris, host cell DNA, and other impurities that have the potential to obstruct filtration processes. Efforts to employ tangential flow filtration (TFF) for initial clarification immediately after harvest have encountered formidable obstacles in managing these high-solids fluids. Deep filtration requires a large volume of water or buffer to equilibrate membrane and generates lots of plastic and liquid waste. Consequently, TFF is more frequently deployed in subsequent purification stages. This highlights the critical necessity for pioneering technical solutions capable of adeptly handling the elevated particulate concentrations encountered in initial process streams.

The notion of continuous media supply holds the promise of enhancing process efficiency and flexibility; however, the current automated systems are primarily tailored to address simpler buffers, rather than the large volume and the complexities of growth media. For example, US20220128523A1 teaches of a computer implemented method for preparing a liquid mixture comprising an acid and its conjugate base or a base and its conjugate acid, but fails to address this need. This unmet need for enabling the continuous preparation and delivery of intricate media at large scales remains an unresolved issue in the industry.

Moreover, distinct challenges arise in the realm of cell-cultured meat production, specifically with regard to the quality control of incoming medium components. These components often exhibit lower and more variable quality in comparison to bio-pharmaceutical-grade raw materials, which can lead to complications during direct flow sterile filtration, such as membrane clogging. Consequently, there is a clear imperative for the development of more resilient and efficient media sterilization methods.

In summation, a palpable and multifaceted demand exists across large-scale bio-manufacturing industries for the introduction of superior technical and economic solutions addressing the challenges posed by media preparation. The proposed invention aims to bridge the gap, overcoming the limitations of current methods by introducing an innovative modular configuration that leverages TFF-based pre-filtration. This disclosure describes groundbreaking innovation is designed to furnish a more robust, flexible, and cost-efficient solution for cell culture media production, all while satisfying the evolving needs of these industries.

### SUMMARY OF THE INVENTION

The following summary is an explanation of some of the general inventive steps for the system, method, devices and apparatus in the description. This summary is not an extensive overview of the invention and does not intend to limit its scope beyond what is described and claimed as a summary.

In some embodiments thereof, the present invention relates to a modular system for efficient preparation and sterile filtration of cell culture media and buffers at large scales. The system comprises a tangential flow filtration (TFF) module, intermediate holding tanks, and a final dilution and sterile filtration module.

According to one aspect, in the TFF module, medium components are mixed at concentrated stock concentrations using smaller volumes versus traditional single batch preparation. The TFF filtration then removes larger particulates and aggregates while filtrating the solution further. TFF enables robust processing of higher solids fluids without membrane detrimental clogging.

The concentrated, pre-filtered medium stock can be held in intermediate bags or tanks for temporary storage and sampling. This allows quality control and adjustments like pH calibration before final preparation.

The stock flows to the final module where it is diluted to desired concentrations using water, recycled media, or buffers. Inline mixing or smaller tanks can be utilized prior to terminal sterile filtration into the bio-reactor.

The multi-step preparation approach leverages TFF's capabilities for handling higher particulate levels, enabling lower quality raw materials while still achieving sterile filtration. The reduced equipment requirements also decrease capital costs compared to traditional batch systems.

Further, the modular configuration can support continuous or semi-continuous media production versus standard batch methods. Process control automation and optimization of intermediate dilution ratios based on TFF rates can enable seamless operations. This provides efficiency benefits for large-scale bio-manufacturing.

In many aspects, the systems disclosed herein offer more robust and flexible preparation of cell culture media and buffers. The inventions ultimately provide significant technical and economic improvements for media supply at large production scales in bio-pharmaceutical and cell-culture meat industries.

### BRIEF DESCRIPTION OF THE DRAWINGS

The novel features believed to be characteristic of the illustrative embodiments are set forth in the appended claims. The illustrative embodiments, however, as well as a preferred mode of use, further objectives and descriptions thereof, will best be understood by reference to the following detailed description of one or more illustrative embodiments of the present disclosure when read in conjunction with the accompanying drawings, wherein:

FIG. 1 illustrates a medium/buffer preparation system with three modules for dissolution and premixing, temporary hold, final dilution & terminal filtration.

### DETAILED DESCRIPTION OF THE PREFERRED EMBODIMENTS

Hereinafter, the preferred embodiment of the present invention will be described in detail with reference to the accompanying drawings. The terminologies or words used in the description and the claims of the present invention should not be interpreted as being limited merely to their common and dictionary meanings. On the contrary, they should be interpreted based on the meanings and concepts of the invention in keeping with the scope of the invention based on the principle that the inventor(s) can appropriately define the terms in order to describe the invention in the best way.

It is to be understood that the form of the invention shown and described herein is to be taken as a preferred embodiment of the present invention, so it does not express the technical spirit and scope of this invention. Accordingly, it should be understood that various changes and modifications may be made to the invention without departing from the spirit and scope thereof.

In this disclosure, the term exemplary may be construed as to mean embodiments that are provided as examples.

In some embodiments thereof, the present invention discloses systems for the preparation of buffers or medium in large volumes that may exhibit themselves in various subsystems including premixing systems, TFF-based stock solution filtration, intermediate holding containers and a final dilution and terminal filtration.

According to one aspect, premixing systems facilitate the initial preparation of medium components, which include elements like powder, water, buffer, and other applicable constituents. The goal is to create concentrated stock solutions. Notably, these premixing systems can be considerably smaller in scale compared to the final volume of the diluted buffer or medium. The premixing process itself can be executed as a batch process, continuous or semi-continuous process.

According to another aspect, the TFF-based stock solution filtration processes stock solutions, which having undergone the initial premixing, are further processed using Tangential Flow Filtration (TFF). The filtration process involves a choice choice of tangential filtration device such as hollow fiber filters and flat sheet cassettes, each capable of using diverse membrane materials, such as PES, PVDF, PTFE, or other materials. The membrane pore sizes can also be tailored to specific requirements, ranging from 0.1 μm for mycoplasma removal to 0.22 μm for sterile filtration, or other molecular weight cut-offs. Post-filtration, additional water, recycled medium, or other components can be introduced into the system, or used for rinsing, to ensure complete component recovery if desired.

In yet another disclosed aspect, intermediate holding containers are discussed, whereby one or more interim hold containers are employed within the system. These containers serve multiple purposes, including quality control, temporary storage, and adjustments to components or pH levels. They can take the form of single-use mixing or storage bags, or stainless-steel tanks. Notably, these hold containers can also incorporate mechanisms for mixing to maintain uniformity and control temperature.

Another aspect is a final dilution and terminal filtration, whereby in the final stages of the process, the prefiltered medium, and process water are combined and subjected to terminal filtration. This step can involve mixing, which can be achieved through the use of an inline mixer or a mixing tank with a much smaller volume compared to the bio-reactor. Terminal filtration can vary in terms of pore size, including options like 0.1 μm or 0.2 μm, or other specified ratings. Additionally, prefiltered recycling medium may also be added to replace a portion or all of the water used in this stage.

The teachings of these collective subsystems form a comprehensive solution to address the preparation of buffers or medium on a large scale, providing flexibility and adaptability to meet varying needs in the field of bio-pharmaceutical and cell-culture production.

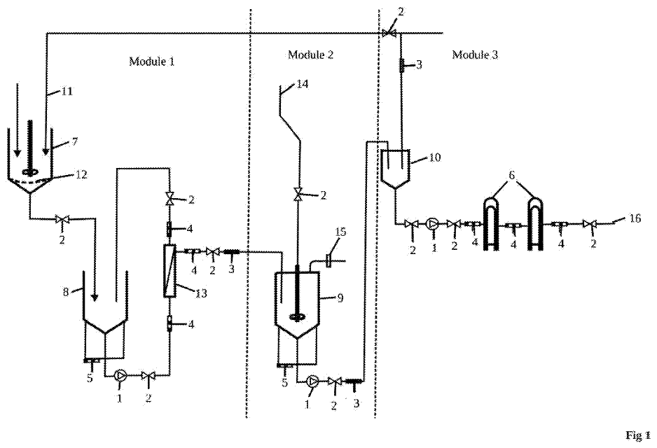

In the illustrated non-limiting embodiment of FIG. 1, a specific application of the invention is detailed to showcase its functionality. This embodiment pertains to a media formulation solution employed in the production of cells via a fed-batch protocol preferably within a 2500 L reactor, boasting a working volume of 2000 L. The operational process is described as follows:

In a non-limiting aspect, the process may commence with the addition of medium dry powder and dilution buffer into a mixing tank (7), where they are combined and dissolved to create a powerful liquid mixture. This mixture can be further added to a tangential flow filtration process tank or reservoir (8), specifically designed for tangential flow pre-filtration. It's noteworthy that the combination of medium powder and buffer can also be carried out in the TFF process reservoir (8) itself. Once the mixture undergoes pre-filtration, the resulting medium concentrate is directed into a buffer or temporary storage tank (9) designated for temporary storage. Importantly, there is an option to partially substitute the medium preparation water or buffer with recovered medium.

Preferably, the medium temporary storage tank (9) is typically implemented as a disposable mixing bag system. Alternatively, it can be substituted with stainless steel or plastic tanks among others, offering flexibility in the system's configuration.

Within this embodiment, quality control analysis is conducted, encompassing essential component adjustments such as pH and osmotic pressure, using the medium temporary storage tank (9). Subsequently, the medium, having passed through the interim check, is subjected to dilution. This dilution process can be executed via a small dilution tank (10). Alternatively, there is the option to incorporate an in-line mixer instead of mixing tank, which can be utilized for simplicity.

The unique attributes of this specific embodiment include the choice of components and equipment tailored to the requirements of a 2500 L reactor. The dissolving and stirring tank (7) may have a volume of 100 L, while the tangential flow filtration system (13) may employ a 100-liter process container (8). The filtration process is preferably executed using 0.2 μm polyether sulfone membrane hollow fiber membrane filters (12), boasting a total surface area of approximately 1.0 m2. This embodiment includes the use of two 100 L disposable mixing bags as temporary storage tanks (9) in parallel, which serve as temporary storage tanks.

The flux of the tangential flow filtration (13) is regulated, preferably aiming for a flow rate of 75 L/m2/hr (LMH). The system is designed to accommodate fluxes as high as 75 LHM or even higher.

When multiple medium temporary storage containers or tanks (9) are employed, these containers may be utilized alternately to supply medium for the final dilution and terminal filtration processes. This configuration has the advantage of ensuring continuous or semi-continuous production, offering a dynamic and efficient operational model.

In some aspect, the dilution process involves the application of a specific ratio, which can be optimized to meet the desired outcomes, as per system design. For instance, the medium to water ratio can be set at 1:4 by volume, which implies that one liter of prefiltered concentrated medium can be diluted to a volume of five liters. The system's design aims to ensure that a capacity of 75 L/hr of pre-filtered medium can meet the final terminal filtration capacity of 375 L/hr in continuous production. This embodiment features a pre-filter system (13) capable of providing 2,250 liters of medium within a 6-hour timeframe. Notably, the membrane area utilized can reach up to 2 square meters, offering a capacity of 2,500 L within 6 hours, sufficient to fill a 3,000 L bioreactor (16) in just 4 hours.

The final dilution filtration process adopts terminal filtration, employing either a single or double layer of 0.2 μm PES membrane (14). This filtration process is achieved through the utilization of a capsule filter or filter element (14), possessing a membrane area of approximately 1 square meter or 20″.

Furthermore, the scalability of the buffer preparation system is emphasized within this embodiment. The technologies employed provide a solution capable of addressing the medium preparation needs for a 200,000 working volume reactor (16). The equipment employed within this configuration includes a dissolving and stirring tank (7) with a volume of 1,000 L, a tangential flow filtration system (13) that utilizes a 1,000-liter disposable mixing bag or tank (9), and 40 square meters of 0.2 μm modified polyether sulfone membrane filters (6). These filters can be designed either in flat sheet cassettes format or s hollow fibers, ceramic membrane, stainless steel filters. The temporary storage tank setup consists of two 1,000 L tanks (9), offering adaptability in the system's layout.

With a throughput of 75 LMH for tangential flow filtration (13), a single system's filtration capability supports a concentrated medium throughput of 3,000 L/hr. The tangential flow filtration process also features a recycle flow of 50 L/min. When multiple medium storage tanks (9) are incorporated, these tanks operate alternately to facilitate the final dilution and filtration procedures, ensuring continuous or semi-continuous production.

The application of filtered pre-filtered media typically adheres to a dilution ratio of 1 to 5. This means that 3,000 L/hr of pre-filtered medium can support a production capacity of approximately 15,000 L/hr of medium, which is then diluted to reach the desired final concentration. The area of the terminal filter (6) may vary but often falls within the range of 20-40 square meters. It typically employs a PES 0.2 μm+0.2 μm double-layer membrane filter element to ensure effective filtration.

Incorporating two such pre-filtration systems enhances the system's capacity significantly. In this scenario, the system is poised to provide over 200,000 liters of medium within an 8-hour timeframe, showcasing its efficiency and scalability in addressing the demands of large-scale processes.

In an alternative embodiment, the preparation process is optimized through a distinct approach. A pre-mixing device takes center stage, where the medium and water are initially blended within this device (7). This mixing and dilution procedure is carried out via batch operations, ensuring that the concentrated medium is effectively diluted. Once the medium is mixed and diluted, it is directed into a process container tank (8) integrated into the tangential flow filtration system (13). An intriguing feature of this embodiment is the potential for the TFF process to function in a continuous mode, provided that the dissolute medium is periodically introduced. This setup streamlines the process by eliminating the need for temporary storage tanks (9), promoting a continuous flow of medium.

In a non-limiting embodiment, it may be provided to the system a control system adapted for regulating the operation of the tangential flow filtration (TFF) module and the final filtration module (6). This control system may be designed to regulate and optimize their output in real-time. For instance, during the pre-filtration process in the TFF module, the control system continuously monitors various parameters such as flow rates, pressure differentials, and other quality attributes of the pre-filtered medium. In some aspects, if it detects deviations from the desired specifications, the control system can automatically adjust the TFF system's (13) operating parameters, such as the filtration rate or transmembrane pressure, to maintain optimal performance.

Also shown in the figure are pumps (1) for transferring fluids between tanks and modules, valves (2) for controlling fluid flow, flow meters (3) for measuring flow rates, pressure sensors (4) for monitoring pressure, load cells (5) for weighing tanks and measuring fluid volumes, terminal filters (6) for removing particulates, vent filter 15 for venting gases while filtering airborne particulates, output line 16 for transferring prepared buffer to the bio-reactor and other components 14 which may include additional valves, sensors, connectors, etc.

In yet another aspect, the control system may interact with external process control systems and sensors to ensure that the final dilution and sterile filtration module's output aligns with the specific needs of the bio-reactor or production process. For instance, it can adapt the dilution ratio based on the processing rate within the TFF module, which may vary due to changes in raw material components or process requirements. Such aspects may be necessary in maintaining the efficiency and reliability of the entire cell culture media preparation system, enabling automated adjustments and optimization to ensure the final output consistently meets the desired standards in large-scale bio-pharmaceutical and cell-cultured meat production.

In an alternative embodiment, emphasis is made on the operational model. In this aspect, the concentrated medium, having undergone tangential flow filtration, is combined directly with filtered water within the reactor. In contrast to the standard configuration that employs a medium concentrate holding tank for temporary storage, this alternative approach bypasses this step. To enhance process safety, a redundant terminal filter can be introduced between the tangential flow filter and the reactor, ensuring an extra layer of filtration before the medium enters the bioreactor. This additional safety measure aligns with quality control practices to further guarantee the medium's integrity.

A notable difference in this alternative embodiment lies in the role of the reactor itself, whereby, instead of relying on separate tanks for temporary storage and pre-dilution processes, the reactor is now used for mixing and quality control of the medium. This innovation streamlines the system by eliminating the need for additional tanks, making it an attractive option for applications where efficiency and simplicity are paramount.

The alternative embodiments present distinct strategies for optimizing the preparation of cell culture media. The first embodiment eliminates the need for temporary storage tanks, promoting a continuous flow of medium through the use of a pre-mixing device. The second embodiment underscores the role of the reactor in the process, allowing for direct mixing of the concentrated medium and filtered water, all while enhancing safety and quality control. These variations in approach offer flexibility and efficiency, catering to diverse production requirements in the bio-pharmaceutical and cell-cultured meat industries.

It should be appreciated that variations and modifications are possible within the spirit and scope of the invention as claimed. Accordingly, the applicant intends to cover reasonable alterations, uses, combinations, and equivalents that align with the underlying inventive concepts disclosed.

It should be noted that reference to singular elements can encompass plural forms, and vice versa, unless explicitly stated or clearly evident from context. Use of grammatical conjunctions is meant to express all conjunctive and disjunctive combinations possibilities, unless indicated otherwise by the context. The term “or” in particular should be understood as generally conveying “and/or”.

### INDUSTRIAL APPLICATION

The present invention has industrial applicability in large-scale cell culture media and buffer preparation systems, particularly designed for biopharmaceutical and cell-cultured meat industries, but also applicable in other industries. The invention aims to streamline and enhance the production of buffer solutions and cell culture media, addressing the challenges associated with traditional methods. By optimizing certain aspects including pre-mixing systems, tangential flow filtration for pre-filtration, intermediate holding tanks, and final dilution and sterile filtration, the invention provides solutions for cost-effective, efficient, and quality-controlled media and buffer preparation, serving the needs of large-scale biomanufacturing processes and high-density cell culture applications, and other related industrial applications.

What is claimed is:

1. A method for preparing cell culture medium or buffer solutions comprising: making and mixing one or more concentrated stock solutions of medium or buffer components in a tangential flow filtration module; pre-filtering the concentrated stock solutions by recirculating through a tangential flow device of the tangential flow filtration module to remove particulates; flowing the pre-filtered concentrated stock solution into one or more intermediate containers; diluting the concentrated stock solution by mixing with water, recycled media, or other diluent; and passing the diluted solution through a sterile filter into a bio-reactor.

2. The method of claim 1, further comprising optimizing a concentration factor in the tangential flow filtration module to reduce processing time to meet other process goals.

3. The method of claim 1, further comprising passing the diluted solution through a redundant sterilizing filter of between 0.05 μm to 0.45 μm before entering the bio-reactor.

4. The method of claim 1, further comprising automatically controlling a dilution ratio proportionate to a processing rate in the tangential flow filtration module.

5. The method of claim 1, further comprising sampling and analyzing the concentrated stock solution in the intermediate holding tanks.

6. The method of claim 1, further comprising continuously recycling solution within the tangential flow filtration module to enhance mixing and filtration.

7. The method of claim 1, further comprising implementing a membrane in the tangential flow filtration module to remove anticipated particulates and impurities from lower quality raw materials.

8. The method of claim 1, further comprising adding water, recycled media, or additional components to the intermediate holding tank or diluted solution.
